(19) United States
(12) Patent Application Publication
(10) Pub. No.: US 2016/0030318 A1
(43) Pub. Date: Feb. 4, 2016

(54) HIGHLY BIOAVAILABLE Na-ACYL ACIDIC AMINO ACID ESTER SALT COMPOSITONS WITH ZWITTERIONIC SURFACTANTS

(71) Applicant: JOHNSON & JOHNSON CONSUMER INC., Skillman, NJ (US)

(72) Inventor: Daniel R. Queiroz, Belle Mead, NJ (US)

(21) Appl. No.: 14/448,505

(22) Filed: Jul. 31, 2014

(51) Int. Cl. A61K 8/43; A61Q 11/00

(52) U.S. Cl. CPC A61K 8/43; A61Q 11/00

(57) ABSTRACT

The present invention provides compositions comprising from about 0.01 to about 3% by weight of the composition of one or more N^{α}-acyl acidic amino acid ester salts represented by the formula I:

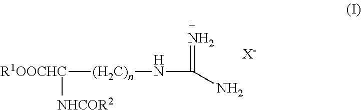

wherein R¹ is an alkyl group having from 1 to 8 carbons, R² is an alkyl group having from 6 to 30 carbons, n is from 1 to 6 and X⁻ is an anion; a zwitterionic surfactant; an alcohol represented by R³—OH, wherein R³ is an alkyl group having from 2 to 6 carbons; an aqueous vehicle; and an oily component, wherein the composition has a pH of less than 7. Also provided are methods of inhibiting plaque in an oral cavity comprising the step of introducing a composition of the present invention into the oral cavity and dispensing devices containing compositions of the present invention.

### FIELD OF THE INVENTION

This invention relates to compositions containing N^{α}-acyl acidic amino acid ester salts having unexpectedly high bioavailability. More specifically, the invention relates to antiplaque and/or anti-gingivitis effective oral care compositions comprising highly bioavailable N^{α}-acyl acidic amino acid ester salts and to methods of inhibiting plaque in the oral cavity using such compositions.

### BACKGROUND OF THE INVENTION

N^{α}-acyl acidic amino acid ester salts have been identified for use in a variety of compositions to provide a variety of benefits, including their use in oral compositions. For example, U.S. Pat. No. 5,266,306 describes an oral composition containing cetylpyridinium chloride as an antibacterial in combination with an N^{α}-cocoyl acyl basic amino acid lower alkyl ester or salt thereof that is said to promote adsorption of cetylpyridinium chloride. Exemplary adsorption promoters include N^{α}-cocoyl-L-arginine methyl ester hydrochloride, N^{α}-cocoyl-L-arginine methyl ester pyrrolidone carboxylate and N^{α}-cocoyl-L-arginine ethyl ester pyrrolidone carboxylate.

GB 1352420 describes lower alkyl esters of mono-N-higher aliphatic acyl arginine having the formula:

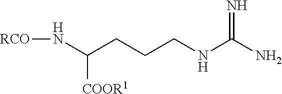

wherein RCO is a higher aliphatic acyl radical containing at least 6 carbon atoms and R¹ is a lower alkyl radical containing up to 4 carbon atoms, or salts of the esters, that have antimicrobial or germicidal activity. In particular it is disclosed that N^{α}-cocoyl-L-arginine ethyl ester pyrrolidone carboxylate and N^{α}-cocoyl-L-arginine methyl ester pyrrolidone carboxylate provide bactericidal activity against Escherichia, Pseudomonas aeruginosa, Proteus vulgarus, Staphylococcus aureus, Bacillus substilis, Candida albicans and Aspergillus niger. It is further asserted that the arginine derivatives provided disinfectant activity against bacterium belonging to the genus Lactobacillus and Staphylococcus. However, this reference neither exemplifies a mouthwash composition nor suggests that the arginine derivatives would be effective against the specific bacterium associated with the formation of plaque.

Arginine esters have been employed in mouthwash compositions as surfactants. JP 04-005221 is directed to mouthwash compositions containing nonwater-soluble components, hydrophilic surfactants, ethanol and water. In particular, this publication describes a mouthwash composition containing vitamin E, N-cocoyl-L-arginine ethyl ester DL-pyrrolidone carboxylate, saccharin, flavor, ethanol and water that is diluted with additional water to give a homogeneous emulsion with an average particle size of 0.44 mm. Such an emulsion would be cloudy. However, mouthwash compositions that are cloudy are disadvantageous since consumers generally require clear single phase mouthwash compositions.

International Publication No. WO 93/11738 describes a dentifrice composition containing a bacteriocin (i.e. an antibacterial agent) such as nisin in combination with a cationic, amphoteric or nonionic surfactant. The D,L-2-pyrrolidone-5-carboxylic acid salt of ethyl-N-cocoyl L arginate is disclosed as a suitable surfactant. JP 57-165305 describes a method for improving the bacteriocidal effect of surfactant type disinfectants such as chlorohexidine digluconate by the addition of N^{α}-cocoyl-L-arginine ethyl ester DL-pyrrolidine carboxylate. The arginine ester is said to have weak bacteriocidal activity, but brings about a synergistic improvement in the bacteriocidal effect of the surfactant type disinfectants.

Aqueous compositions containing salts of N^{α}-alkyl-L-arginine alkyl esters generally undergo hydrolysis reactions typical of esters. U.S. Pat. No. 5,874,068 discloses antiplaque and antigingivitis effective oral compositions containing stabilized N^{α}-alkyl-L-arginine alkyl ester salts useful as disinfectants or bactericidally active ingredients effective against the specific microorganisms associated with plaque buildup. The invention is also directed to a method for inhibiting plaque in the oral cavity.

U.S. 2010/0330136A1 relates to the use of cationic surfactants with antimicrobial properties for protecting teeth and the oral cavity. The most preferred compound of the above class of cationic surfactant is the ethyl ester of the lauramide of the arginine monohydrochloride (CAS No. 60372-77-2). Examples of the composition are in the form of a chewing gum or a lozenge.

Applicants have nevertheless recognized the need for compositions having increased bioavailability of N^{α}-acyl acidic amino acid ester salts therein.

### SUMMARY OF THE INVENTION

Applicants have discovered unexpectedly that N^{α}-acyl acidic amino acid ester salts can be combined with zwitterionic surfactants to form compositions having relatively higher bioavailability of the N^{α}-acyl acidic amino acid ester salts therein. Such compositions are particularly suitable for oral applications, including, but not limited to, oral compositions for inhibiting plaque.

In one aspect, the present invention provides compositions comprising from about 0.01 to about 3% by weight of the composition of one or more N^{α}-acyl acidic amino acid ester salts represented by the formula I:

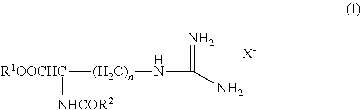

wherein R¹ is an alkyl group having from 1 to 8 carbons, R² is an alkyl group having from 6 to 30 carbons, n is from 1 to 6 and X⁻ is an anion; a zwitterionic surfactant; an alcohol represented by R³—OH, wherein R³ is an alkyl group having from 2 to 6 carbons; an aqueous; and an oily component, wherein the composition has a pH of less than 7.

The invention is also directed to a method of inhibiting plaque in an oral cavity comprising the step of introducing a composition of the present invention into the oral cavity.

### DESCRIPTION OF PREFERRED EMBODIMENTS

All percentages listed in this specification are percentages of solids/active amounts by weight, unless otherwise specifically mentioned.

Without intending to be limited to theory, applicants have recognized that N^{α}-acyl acidic amino acid ester salts can be used to modify the surface of the teeth to minimize microbial adhesion, and that the compositions of the present invention unexpectedly tend to exhibit higher bioavailability of such salts for use in oral, or other, compositions. In particular, applicants note that, in the mouth, saliva generates a pellicle that forms on the tooth surface. This salivary pellicle is primarily composed of proteinaceous material that forms a coating on the tooth surface allowing bacteria to adhere. The formation of bacterial plaque is initiated by the adhesion of microorganisms to the tooth surface, and is the first step in the potential development of gingivitis. It has been recognized that a valid way to prevent plaque formation may be to interfere with adhesion of bacteria to the tooth surface. In vitro studies have shown that hydrophobic surfaces minimize bacteria attachment/adhesion thereby reducing biofilm development and bacteria growth.

Applicants have recognized that the preventative effect described above can be achieved via adsorption of salts of N^{α}-acyl acidic amino acid ester onto the tooth surface. Such salts bound to the pellicle may function as a physical barrier to bacterial adhesion on the teeth surfaces. The barrier function to bacterial adhesion may occurs through two way: (i) the physical presence of the salt material on the salivary pellicle surface prevents the adhesion of bacteria to the pellicle, and (b) the adhesion of the salt material to the pellicle also reduces the surface energy of the tooth surface.

Any of a wide variety of N^{α}-acyl acidic amino acid ester salts are suitable for use in the compositions of the present invention. In particular, any one or more N^{α}-acyl acidic amino acid ester salts of formula I are suitable for use herein:

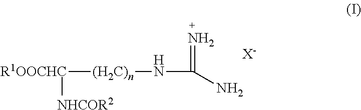

wherein R¹ is an alkyl group having from 1 to 8 carbons, R² is an alkyl group having from 6 to 30 carbons, n is from 1 to 6 and X⁻ is an anion. Examples of suitable salts of Formula I including salts wherein the anion X⁻ is chloride, or DL-pyrrolidone carboxylate.

In some embodiments, the N^{α}-acyl acidic amino acid ester salts employed in this invention are the salts of N^{α}-cocoyl arginine ethyl ester, N^{α}-lauryl arginine ethyl ester, N^{α}-lauryl arginine iso-propyl ester, or combinations of two or more thereof. In certain embodiments, the N^{α}-acyl acidic amino acid ester salts are selected from the group consisting of chloride salts of N^{α}-cocoyl arginine ethyl ester, N^{α}-lauryl arginine ethyl ester, and N^{α}-lauryl arginine iso-propyl ester; DL-pyrrolidone carboxylate salts of N^{α}-cocoyl arginine ethyl ester, N^{α}-lauryl arginine ethyl ester, and N^{α}-lauryl arginine iso-propyl ester; and combinations of two or more thereof. In certain embodiments, the N^{α}-acyl acidic amino acid ester salt is, or comprises, the chloride salt of N^{α}-lauryl arginine ethyl ester (LAE).

In some embodiments, the N^{α}-acyl acidic amino acid ester salts employed in this invention are the salts of N^{α}-cocoyl-L-arginine ethyl ester, N^{α}-lauryl-L-arginine ethyl ester, N^{α}-lauryl-L-arginine iso-propyl ester, or combinations of two or more thereof. In certain embodiments, the N^{α}-acyl acidic amino acid ester salts are selected from the group consisting of chloride salts of N^{α}-cocoyl-L-arginine ethyl ester, N^{α}-lauryl-L-arginine ethyl ester, and N^{α}-lauryl-L-arginine iso-propyl ester; DL-pyrrolidone carboxylate salts of N^{α}-cocoyl-L-arginine ethyl ester, N^{α}-lauryl-L-arginine ethyl ester, and N^{α}-lauryl-L-arginine iso-propyl ester; and combinations of two or more thereof. In certain embodiments, the N^{α}-acyl acidic amino acid ester salt is, or comprises, the chloride salt of N^{α}-lauryl-L-arginine ethyl ester (LAE).

Any suitable amount of N^{α}-acyl acidic amino acid ester salts of Formula I may be used in the compositions of the present invention. In certain embodiments, the salt is present in the composition in an amount that is effective to inhibit plaque buildup in the oral cavity and that is stable in the oral composition. Generally, the composition contains a total amount of N^{α}-acyl acidic amino acid ester salt(s) in an amount from 0.001% (or about 0.001%) to 3% (or about 3%) w/v of the composition. In some embodiments, the N^{α}-acyl acidic amino acid ester salt is present in an amount from 0.01% (or about 0.01%) to 3% (or about 3%) w/v of the composition, 0.01% (or about 0.01%) to 0.6% (or about 0.6%) w/v of the composition, or from 0.1% (or about 0.1%) to 3% (or about 3%) w/v of the composition or from 0.1% (or about 0.1%) to 0.3% (or about 0.3%) w/v of the composition.

Any of a variety of zwitterionic surfactants may be used in the composition of the claimed invention. Examples of zwitterionic surfactants that may be used include betaine surfactants and those disclosed in U.S. Pat. No. 5,180,577, incorporated herein by reference. In certain embodiments, the zwitterionic surfactant is selected from the group consisting of alkyldimethyl betaines, including but not limited to, decyl betaine, cocobetaine, myristyl betaine, palmityl betaine, lauryl betaine, cetyl betaine, stearyl betaine, combinations of two or more thereof and the like; and amidobetaines, including but not limited to, cocamidoethyl betaine, cocamidopropyl betaine, laurmidopropyl betaine and combinations of two or more thereof. In certain embodiments, the zwitterionic surfactant is selected from the group consisting of decyl betaine, cocobetaine, myristyl betaine, palmityl betaine, lauryl betaine, cetyl betaine, stearyl betaine, cocamidoethyl betaine, cocamidopropyl betaine, laurmidopropyl betaine, and combinations of two or more thereof. In certain embodiments, the zwitterionic surfactant is selected from the group consisting of cocamidopropyl betaine, lauramidopropyl betaine, cetyl betaine, and combinations of two or more thereof.

Any suitable amount of zwitterionic surfactant may be used in the compositions of the present invention. Generally, the composition contains a total amount of zwitterionic surfactant 0.01% (or about 0.01%) to 8% (or about 8%) w/v of the composition. In some embodiments, the Zwitterionic surfactant is present in an amount from 0.01% (or about 0.01%) to 2% (or about 2%) w/v of the composition, from 0.1% (or about 0.1%) to 2% (or about 2%) w/v of the composition, or from 0.1% (or about 0.1%) to 1% (or about 1%) w/v of the composition.

Any of a variety of alcohols represented by the formula R³—OH, wherein R³ is an alkyl group having from 2 to 6 carbons, may be used in the present invention. Examples of suitable alcohols of formula R³—OH include ethanol; n-propanol, iso-propanol; butanols; pentanols; hexanols, and combinations of two or more thereof, and the like. Preferably the alcohol is selected such that R³ is the same as R¹ in the molecule of formula I used in the same composition. In certain embodiments, the alcohol is, or comprises, ethanol.

Any suitable amount of alcohol of formula R³—OH may be used in the present invention. The alcohol is present in an amount that inhibits the hydrolysis of the ester of the N^{α}-acyl acidic amino acid ester salt, thereby stabilizing the compound. The alcohol may be present in the composition in an amount of at least 10.0% (or about 10.0%) v/v of the total composition, or from 10.0% (or about 10.0%) to 35.0% (or about 35.0%) v/v of the total composition, or from 15.0% (or about 15.0%) to 30.0% (or about 30.0%) v/v of the total composition and may be from 20.0% (or about 20.0%) to 25.0% (or about 25.0%) v/v of the total composition.

The compositions of the present invention preferably have a pH of less than 7. In certain embodiments, the composition have a pH of from 3.0 (or about 3.0) to less than 7.0 (or less than about 7.0), or from 3.5 (or about 3.5) to 6.5 (or about 6.5), or from 3.5 (or about 3.5) to 5.0 (or about 5.0).

As will be recognized by those of skill in the art, the pH of the composition may be adjusted or achieved using a buffer in an amount effective to provide the composition with a pH below 7. The composition can optionally comprise at least one pH modifying agents among those useful herein include acidifying agents to lower pH, basifying agents to raise pH, and buffering agents to control pH within a desired range. For example, one or more compounds selected from acidifying, basifying and buffering agents can be included to provide a pH of about 2 to about 7, or in various embodiments from about 3 to about 6, or from about 4 to about 5. Any orally acceptable pH modifying agent can be used including without limitation carboxylic and sulfonic acids, acid salts (e.g., monosodium citrate, disodium citrate, monosodium malate, etc.), alkali metal hydroxides such as sodium hydroxide, borates, silicates, imidazole and mixtures thereof. One or more pH modifying agents are optionally present in a total amount effective to maintain the composition in an orally acceptable pH range. In certain embodiments, inorganic acids may be used as the buffer added to the composition.

In certain embodiments, organic acids may be used as the buffer added to the composition. Organic acids suitable for use in the compositions of the present invention include, but are not limited to, ascorbic acid, sorbic acid, citric acid, glycolic acid, lactic acid and acetic acid, benzoic acid, salicylic acid, phthalic acid, phenolsulphonic acid, succinic acid and mixtures thereof, optionally, the organic acid is selected from the group consisting of benzoic acid, sorbic acid, succinic acid, citric acid and mixtures thereof, or optionally, the organic acid is benzoic acid.

Generally the amount of acidic buffer is between about 0.001% (or about 0.001% w/v) to about 5.0% (or about 5.0% w/v) of the composition. In certain embodiment, the organic acid buffer is present in amounts of from 0.001% (or about 0.001% w/v) to 1.0% w/v (or about 1.0% w/v) of the composition, or between about 0.100% (or about 0.100% w/v) to about 1.0% (or about 1.0% w/v) of the composition.

The composition also includes an aqueous vehicle. The aqueous vehicle is generally water, although water/alcohol mixtures may also be employed. In certain embodiments, water is added to q.s. (Quantum Sufficit, Latin for “as much as needed”) the composition. In certain embodiments, the aqueous phase comprises from about 60% to about 95%, or from about 75% to about 93%, by weight of the composition. In certain compositions, water is present in an amount of from about 60% to about 95%, or from about 75% to about 93%. Alternatively, the compositions of the present invention may be formulated in a dry powder, chewing gum, film, semi-solid, solid or liquid concentrate form. In such embodiments, for example, water is added to q.s. as necessary in the case of liquid concentrates or powdered formulations, or water may be removed using standard evaporation procedures known in the art to produce a composition in dry powder form. Evaporated, or freeze dried forms are advantageous for storage and shipping.

Any of a variety of oily components may be used in the present compositions. The oily component may comprise any one or more oils, or other materials that are water-insoluble, or substantially water-insoluble, meaning that its solubility is less than about 1% by weight in water at 25° C. or, optionally, less than about 0.1%. In certain embodiments, the oily component of the present invention comprises, consists essentially of, or consists of, at least one essential oil, i.e. a natural or synthetic (or combination thereof) concentrated hydrophobic material of vegetable origin, generally containing volatile compounds.

at least one flavor oil, or a combination of two or more thereof. Examples of suitable essential oils, flavor oils, and their amounts are described below. In certain embodiments, the composition comprises a total amount of oily component of at least 0.05% w/w (or about 0.05%), at least 0.1% w/w (or about 0.1% w/w), or at least 0.2% w/w (or about 0.2% w/w) of oily component.

In certain embodiments, essential oils are also added to the oral compositions of the present invention. (e.g. thymol, eucalyptol, menthol, methyl salicylate and/or isomers of each of these compounds).

Thymol, [(CH₃)₂CHC₆H₃(CH₃)OH, also known as isopropyl-m-cresol], is only slightly soluble in water but is soluble in alcohol, and its presence is one of the reasons alcohol was necessary in the well-established, high alcohol commercial mouth rinses. Methyl salicylate, [C₆H₄OHCOOCH₃, also known as wintergreen oil], additionally provides flavoring to the together with its antimicrobial function. Eucalyptol (C₁₀H₁₈O, also known as cineol) is a terpene ether and provides a cooling, spicy taste. Eucalyptol may be used in place of thymol in certain formulations in the same amount if desired. Menthol (CH₃C₆H₉(C₃H₇)OH), also known as hexahydrothymol) is also only slightly soluble in alcohol, and is fairly volatile. Menthol, in addition to any antiseptic properties, provides a cooling, tingling sensation.

In certain embodiments, the essential oils are used in amounts effective to provide antimicrobial activity in the oral cavity. In specific embodiments, the total amount of essential oils present in the disclosed compositions can be from 0.001% (or about 0.001%) to 0.35% (or about 0.35%) w/v, or from 0.16% (or about 0.16%) to 0.28% (or about 0.28%) w/v of the composition.

In some embodiments, the compositions of the present invention contain thymol, eucalyptol, menthol, methyl salicylate, or mixtures of two or more thereof. In some embodiments, the composition contains all four of these essential oils.

In certain embodiments, thymol is employed in amounts of from 0.001% (or about 0.001%) to 0.25% (or about 0.25%) w/v, or from 0.04% (or about 0.04%) to 0.07% (or about 0.07%) w/v of the composition. In certain embodiments, eucalyptol may be employed in amounts of from 0.001% (or about 0.001%) to 0.11% (or about 0.11%) w/v, or from 0.085% (or about 0.085%) to 0.10% (or about 0.10%) w/v of the composition. In certain embodiments, menthol is employed in amounts of from 0.001% (or about 0.001%) to 0.25% (or about 0.25%) w/v, or from 0.035% (or about 0.035%) to 0.05% (or about 0.05%) w/v of the composition. In certain embodiments, methyl salicylate is employed in amounts of from 0.001% (or about 0.001%) to 0.08% (or about 0.08%) w/v, or from 0.04% (or about 0.04%) to 0.07% (or about 0.07%) w/v of the composition.

In certain embodiments, the composition further comprises flavors or flavorants to modify or magnify the taste of the composition, or reduce or mask the sharp “bite” or “burn” of ingredients such as thymol. Suitable flavors include, but are not limited to, flavor oils such as oil of anise, anethole, benzyl alcohol, spearmint oil, citrus oils, vanillin and the like may be incorporated. Other flavors such as citrus oils, vanillin and the like may be incorporated to provide further taste variations. In these embodiments, the amount of flavor oil added to the composition can be from 0.001% (or about 0.001%) to 1.0% (or about 1.0%) w/v, or from 0.01% (or about 0.010%) to 0.30% (or about 0.30%) w/v of the total composition. The particular flavors or flavorants, and other taste-improving ingredients, employed will vary depending upon the particular taste and feel desired. Those skilled in the art can select and customize these types of ingredients to provide the desired results.

In certain embodiments, the compositions of the present invention also comprise less than 0.24% by weight of the composition of non-ionic surfactant. Those of skill in the art will recognized that any of a variety of one or more non-ionic surfactants include, but are not limited to, compounds produced by the condensation of alkylene oxide groups (hydrophilic in nature) with an organic hydrophobic compound which may be aliphatic or alkyl-aromatic in nature. Examples of suitable nonionic surfactants include, but are not limited to, alkyl polyglucosides; alkyl glucose amines, block copolymers such as ethylene oxide and propylene oxide copolymers e.g. Poloxamers; ethoxylated hydrogenated castor oils available commercially for example under the trade name CRODURET (Croda Inc., Edison, N.J.); Alkyl polyethylene oxide e.g. Polysorbates, and/or; fatty alcohol ethoxylates; polyethylene oxide condensates of alkyl phenols; products derived from the condensation of ethylene oxide with the reaction product of propylene oxide and ethylene diamine; ethylene oxide condensates of aliphatic alcohols; long chain tertiary amine oxides; long chain tertiary phosphine oxides; long chain dialkyl sulfoxides; and mixtures thereof.

Exemplary non-ionic surfactants are selected from the group known as poly(oxyethylene)-poly(oxypropylene) block copolymers. Such copolymers are known commercially as poloxamers and are produced in a wide range of structures and molecular weights with varying contents of ethylene oxide. These non-ionic poloxamers are non-toxic and acceptable as direct food additives. They are stable and readily dispersible in aqueous systems and are compatible with a wide variety of formulations and other ingredients for oral preparations. These surfactants should have an HLB (Hydrophilic-Lipophilic Balance) of between about 10 and about 30 and preferably between about 10 and about 25. By way of example, non-ionic surfactants useful in this invention include the following poloxamers:

105
108
124
184
185
188
215
217
234
235
237
238
284
288
333
334
335
338
407

In certain embodiments, the compositions of the claimed invention comprise less than about 2% of non-ionic surfactant, less than 2%, or less than 1.5%, or less than 1%, or less than 0.5, less than 0.24%, less than 0.2%, or less than 0.1% of non-ionic surfactants. In certain embodiments, the composition of the present invention is free of non-ionic surfactants.

Applicants have recognized that compositions of the present invention tend to exhibit unexpectedly higher bioavailability of the N^{α}-acyl acidic amino acid ester salts as compared to comparable compositions known in the art. In certain embodiments, the compositions exhibit a percent bioavailability (as measured using the Disc Retention Assay (DRA) described herein) of greater than about 50%, or greater than about 75%, or greater than about 90%.

Other Ingredients

In certain embodiments, a sugar alcohol (humectant) is also added to the oral compositions of the present invention. The sugar alcohol solvent(s) may be selected from those multi-hydroxy-functional compounds that are conventionally used in oral and ingestible products. In certain embodiments, the sugar alcohol (s) should be non-metabolized and non-fermentable sugar alcohol (s). In specific embodiments, the sugar alcohols include, but are not limited to sorbitol, glycerol, xylitol, mannitol, maltitol, inositol, allitol, altritol, dulcitol, galactitol, glucitol, hexitol, iditol, pentitol, ribitol, erythritol and mixtures thereof. Optionally, the sugar alcohol is selected from the group consisting of sorbitol and xylitol or mixtures thereof. In some embodiments, the sugar alcohol is sorbitol. In certain embodiments, the total amount of sugar alcohol (s), which are added to effectively aid in the dispersion or dissolution of the mouth rinse or other ingredients, should not exceed 20% w/v (or about 20% w/v) of the total composition. Or, total amount of sugar alcohol should not exceed 17% w/v (or about 17% w/v) of the total composition. Or, total amount of sugar alcohol should not exceed 10% w/v (or about 10% w/v) of the total composition. The sugar alcohol can be in an amount of from 1.0% (or about 1.0%) to 20.0% (or about 20.0%) w/v, or from 2.5% (or about 2.5%) to 17.0% (or about 17.0%) w/v, or from 5.0% (or about 5.0%) to 15.0% (or about 15.0%) w/v of the total composition.

In certain embodiments, a polyol solvent is added to the composition. The polyol solvent comprises a polyol or polyhydric alcohol selected from the group consisting of polyhydric alkanes (such as propylene glycol, glycerin, butylene glycol, hexylene glycol, 1,3-propanediol); polyhydric alkane esters (dipropylene glycol, ethoxydiglycol); polyalkene glycols (such as polyethylene glycol, polypropylene glycol) and mixtures thereof. In certain embodiments, the polyol solvent can be present in an amount of from 0% to 20.0% (or about 20.0%) w/v, or from 0.5% (or about 0.5%) to 15.0% (or about 15.0%) w/v, or from 2.5% (or about 2.5%) to 8.0% (or about 8.0%) w/v of the composition.

Sweeteners such as aspartame, sodium saccharin, sucralose, stevia, acesulfame K and the like may be added for better taste in amounts of from about 0.001% w/v to about 1.0% w/v. In certain preferred embodiments, the sweetener comprises sucralose.

### Optional Ingredients

### Fluoride Releasing Compounds

In certain embodiments, fluoride providing compounds may be present in the mouth rinse compositions of this invention. These compounds may be slightly water soluble or may be fully water soluble and are characterized by their ability to release fluoride ions or fluoride containing ions in water. Typical fluoride providing compounds are inorganic fluoride salts such as soluble alkali metal, alkaline earth metal, and heavy metal salts, for example, sodium fluoride, potassium fluoride, ammonium fluoride, cupric fluoride, zinc fluoride, stannic fluoride, stannous fluoride, barium fluoride, sodium hexafluorosilicate, ammonium hexafluorosilicate, sodium fluorozirconate, sodium monofluorophosphate, aluminum mono- and difluorophosphate and fluorinated sodium calcium pyrophosphate. Amine fluorides, such as N′-octadecyltrimethylendiamine-N,N,N′-tris(2-ethanol)-dihydrofluoride and 9-octadecenylamine-hydrofluoride), may also be used.

In certain embodiments, the fluoride providing compound is generally present in an amount sufficient to release up to 0.15% (or about 0.15%), or 0.001% (or about 0.001%) to 0.1% (or about 0.1%), or from 0.001% (or about 0.001%) to 0.05% (or about 0.05%) fluoride by weight of the composition.

### Zinc Salts

In certain embodiments, zinc salts such as zinc chloride, zinc acetate or zinc citrate may be added as an astringent for an “antiseptic cleaning” feeling, as a breath protection enhancer or as anticalculus agent in an amount of from 0.0025% w/v (or about 0.0025% w/v) to 0.75% w/v (or about 0.75% w/v) of the composition.

### Sensitivity Reducing Agents

In certain embodiments, sensitivity reducing agents, namely potassium salts of nitrate and oxalate in an amount from 0.1% (or about 0.1%) to 5.0% (or about 5.0%) w/v of the composition may be incorporated into the present invention. Other potassium releasing compounds are feasible (e.g. KCl). High concentrations of calcium phosphates may also provide some added sensitivity relief. These agents are believed to work by either forming an occlusive surface mineral deposit on the tooth surface or through providing potassium to the nerves within the teeth to depolarize the nerves. A more detailed discussion of suitable sensitivity reducing can be found in US 20060013778 to Hodosh and U.S. Pat. No. 6,416,745 to Markowitz et al., both of which are herein incorporated by reference in their entirety.

### Anticalculus Agents

In certain embodiments, compounds with anti-calculus benefits (e.g. various carboxylates, polyaspartic acid, etc.) may be incorporated into the present invention. Also useful as an anticalculus agent are the anionic polymeric polycarboxylates. Such materials are well known in the art, being employed in the form of their free acids or partially or preferably fully neutralized water soluble alkali metal (e.g. potassium and preferably sodium) or ammonium salts. Preferred are 1:4 to 4:1 by weight copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and preferably S-97 Pharmaceutical Grade (M.W. 70,000), of GAF Chemicals Corporation.

### Additional Ingredients

Although the liquid or mouth rinse compositions of the present invention may be formulated to be substantially clear and/or colorless to the unaided eye, acceptably approved food dyes are preferably used to provide a pleasing color to the compositions of the invention. These may be selected from, but not limited to, the long list of acceptable food dyes. Suitable dyes for this purpose include FD&C yellow #5, FD&C yellow #10, FD&C blue #1 and FD&C green #3. These are added in conventional amounts, typically in individual amounts of from 0.00001% w/v (or about 0.00001% w/v) to 0.0008% w/v (or about 0.0008% w/v), or from 0.00035% w/v (or about 0.00035% w/v) to 0.0005% w/v (or about 0.0005% w/v) of the composition.

Other conventional ingredients may be used in the liquid or mouth rinse compositions of this invention, including those known and used in the art. Examples of such ingredients include thickeners, suspending agents and softeners. Thickeners and suspending agents useful in the compositions of the present invention can be found in U.S. Pat. No. 5,328,682 to Pullen et al., herein incorporated by reference in its entirety. In certain embodiments, these are incorporated in amounts of from 0.1% w/v (about 0.1% w/v) to 0.6% w/v (or about 0.6% w/v), or 0.5% w/v (or about 0.5% w/v) of the composition.

A more detailed description of useful oral care actives and/or inactive ingredients and further examples thereof can be found in U.S. Pat. No. 6,682,722 to Majeti et al. and 6,121,315 to Nair et al., each of which are herein incorporated by reference in its entirety.

In certain embodiments, the compositions of the present invention are free of or essentially free of bioavailability affecting compounds. As used herein, “bioavailability affecting compound”, means compounds that negatively affect the bioavailability of any incorporated essential oils N^{α}-acyl acidic amino acid ester salts represented by the formula I such as by binding or otherwise inactivating such N^{α}-acyl acidic amino acid ester salts represented by the formula I. “Essentially free” as used with respect to bioavailability affecting compounds is defined as formulations having less than 5% (or about 5%), or less than 3% (or about 3%), or less than 1% (or about 1%), or less than 0.1, or less than 0.01% (or about 0.01%), by weight (w/v) of the total composition of a bioavailability affecting compound.

The compositions of the present invention may be used as, or in, any of a variety of products for use in the oral cavity. For example, in certain embodiments, the compositions of the present invention are in the form of a mouthwash, mouthrinse, toothpaste, gel, solutions, mousse, foam, denture care product, mouth spray, lozenge or chewable tablet. In certain embodiments, the composition is in the form of a mouthwash or mouthrinse.

### Methods of Practicing the Present Invention

Also provided are methods of inhibiting plaque in an oral cavity comprising the step of introducing a composition of the present invention into the oral cavity in an amount effective and for a time sufficient to prevent plaque accumulation and therefore gingivitis by preventing the adhesion of the microorganisms associated with plaque buildup.

In certain embodiments, the compositions of the present invention are applied to teeth and/or soft surfaces of the oral cavity for at least two consecutive applications, or, at least (or greater than) 3 (or about 3), or at least (or greater than) 5 (or about 5) consecutive applications.

When applied to teeth and/or soft surfaces of the oral cavity, in certain embodiments, the composition is allowed to remain in contact with the teeth and/or soft surfaces of the oral cavity for at least (or greater than) 10 (or about 10) seconds, or 20 (or about 20) seconds, or 30 (or about 30) seconds, or 50 (or about 50) seconds, or 60 (or about 60) seconds.

Various embodiments of the invention have been set forth above. Each embodiment is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents.

Concentrated Compositions

The present invention further provides for concentrated compositions comprising one or more N′-acyl acidic amino acid ester salts represented by the formula I as defined above, zwitterionic surfactant, and alcohol represented by R³—OH, wherein R³ is an alkyl group having from 2 to 6 carbons. Such concentrated compositions may include any of the N^{α}-acyl acidic amino acid ester salts, zwitterionic surfactants, alcohol, and oily components as described above, as well as, any of the buffers, oily components, surfactants, other ingredients, including sugar alcohol, polyol solvent and/or sweeteners as described above, and optional ingredients as described above, but such concentrated compositions will comprise lower amounts of water than the previously described compositions comprising an aqueous vehicle. Preferably, the concentrated compositions comprise the materials in amounts and ratios such that the addition of water thereto will result in a composition of the present invention comprising an aqueous vehicle as described above.

According to certain embodiments, the concentrated compositions of the present invention comprise the N^{α}-acyl acidic amino acid ester salt in an amount from 0.025% (or about 0.025%) to 7.5% (or about 7.5%) w/v of the composition, 0.025% (or about 0.025%) to 1.5% (or about 1.5%) w/v of the composition, or from 0.25% (or about 0.25%) to 7.5% (or about 7.5%) w/v of the composition or from 0.25% (or about 0.25%) to 0.75% (or about 0.75%) w/v of the composition. As described above, in certain embodiments, the N^{α}-acyl acidic amino acid ester salts are selected from the group consisting of chloride salts of N^{α}-cocoyl arginine ethyl ester, N^{α}-lauryl arginine ethyl ester, and N^{α}-lauryl arginine iso-propyl ester; DL-pyrrolidone carboxylate salts of N^{α}-cocoyl arginine ethyl ester, N^{α}-lauryl arginine ethyl ester, and N^{α}-lauryl arginine iso-propyl ester; and combinations of two or more thereof. In certain embodiments, the N^{α}-acyl acidic amino acid ester salt is, or comprises, the chloride salt of N^{α}-lauryl arginine ethyl ester (LAE).

In certain embodiments, the concentrated compositions contain a total amount of zwitterionic surfactant of 0.025% (or about 0.025%) to 20% (or about 20%) w/v of the composition. In some embodiments, the Zwitterionic surfactant is present in an amount from 0.025% (or about 0.025%) to 5% (or about 5%) w/v of the composition, from 0.25% (or about 0.25%) to 5% (or about 5%) w/v of the composition, or from 0.25% (or about 0.25%) to 2.5% (or about 2.5%) w/v of the composition. Examples and preferred zwitterionic surfactants are as described above.

In certain embodiments, the concentrated compositions contain alcohol of formula R³—OH in an amount of at least 40% (or about 40%) v/v of the total composition, or at least 45% (or about 45%) v/v of the total composition, or at least 50% (or about 50%) v/v of the total composition, or at least 60% (or about 60%) v/v of the total composition, or at least 70% (or about 70%) v/v of the total composition, or from 35% (or about 35%) to 75% (or about 75%) v/v of the total composition and may be from 45% (or about 45%) to 65% (or about 65%) v/v of the total composition. Examples and preferred alcohols of formula R³—OH are as described above.

In certain embodiments, the composition comprises a total amount of oily component of at least 0.125% w/w (or about 0.125%), at least 0.25% w/w (or about 0.25% w/w), or at least 0.5% w/w (or about 0.5% w/w) of oily component. In certain preferred embodiments, the oily component of the concentrated composition comprises essential oils in an amount of from 0.0025% (or about 0.0025%) to 0.875% (or about 0.875%) w/v, or from 0.4% (or about 0.4%) to 0.7% (or about 0.7%) w/v of the composition. In certain preferred embodiments, the oily component of the concentrated composition comprises flavor oils in an amount of from 0.0025% (or about 0.0025%) to 2.5% (or about 2.5%) w/v, or from 0.025% (or about 0.025%) to 0.75% (or about 0.75%) w/v of the total composition. Examples of suitable and preferred oily components, including essential oils and/or flavor oils are as described above.

In certain embodiments, the concentrated compositions of the claimed invention comprise less than about 5% of non-ionic surfactant, less than 5%, or less than 3.75%, or less than 2.5%, or less than 1.25, less than 0.6%, less than 0.5%, or less than 0.25% of non-ionic surfactants. In certain embodiments, the composition of the present invention is free of non-ionic surfactants.

In certain embodiments, the concentrated compositions will comprise less than 30% v/v of water, or less than 20% v/v of water, or less than 10% v/v of water, or less than 5% v/v of water. In certain embodiments the concentrated compositions are free of water.

The concentrated compositions of the present invention may be used in any form of product as disclosed above, including, in the form of a mouthwash or mouthrinse or a concentrate thereof, as well as, a toothpaste, gel, solutions, mousse, foam, denture care product, mouth spray, lozenge or chewable tablet. In certain embodiments, the composition is in the form of a mouthwash or mouthrinse concentrate. In certain other embodiments the composition is in the form of a mouth spray.

In certain embodiments, a mouthwash concentrate or mouthrinse concentrate may be used to form a more dilute mouthwash or mouthrinse composition by adding the concentrate to a liquid carrier comprising, or consisting of water, or vice versa. In certain embodiments, the present invention includes a method of making a mouthwash or mouthrinse comprising combining a concentrated composition of the present invention with a liquid carrier comprising water. The present invention also provides methods of inhibiting plaque in an oral cavity comprising the step of combining a concentrated composition of the present invention with a liquid carrier comprising water to form a diluted composition and introducing the diluted composition into the oral cavity.

With respect to the use of a composition as a mouth spray, the present invention comprises a method of treating an oral cavity by spraying a composition of the present invention into the oral cavity. In certain embodiments, the present invention comprises a mouth spray device containing a composition of the present invention and capable of spraying the composition into the oral cavity of a user.

Any device suitable for containing and spraying a composition of the present invention may be used. For example, any device comprising a containing for containing a composition of the present invention and a dispensing assembly associated with the container for dispensing the composition from the device through an orifice (for example toward or into a user's oral cavity when activated by a user) would be suitable in certain embodiments. Examples of dispensing assemblies suitable for use include pump spraying assemblies, including manual pump spraying assemblies, pressure pump sprayers, assemblies using one or more aerosols to dispense the composition, and the like. Specific examples of devices, containers, and spray assemblies suitable for use in the present invention are disclosed in U.S. Pat. Nos. 7,637,394, 7,651,009, and 8,261,946, the specifications of which are incorporated herein by reference in the entireties.

This invention will be better understood from the experimental details that follow. However, one skilled in the art will readily appreciate that the specific method and results discussed are merely illustrative of the invention and no limitation of the invention is implied.

### EXAMPLES

### Example 1

A mouthrinse composition was initially prepared having composition shown in Table 1.

| | | Quantity in | Quantity in |
|---|---|---|---|
| | | finished | finished |
| | | product | product |
| Component | Function | (% w/w) | (% w/v) |
| Purified Water USP | Vehicle | 60.949 | QS |
| Sorbitol Solution 70% | Humectant | 19.627 | 20.000 |
| USP | | | |
| Ethanol USP (190 proof) | Solvent | 18.073 | 18.417 |
| Flavor -Mint | Flavor | 0.144 | 0.146 |
| Poloxamer 407 NF | Surfactant | 0.245 | 0.250 |
| Ethyl Lauroyl Arginate | Anti-plaque | 0.147 | 0.150 |
| HCl (LAE)* | adhesion agent | | |
| Benzoic Acid USP | Buffering Agent | 0.118 | 0.120 |
| Sucralose NF | Sweetener | 0.059 | 0.060 |
| Sodium Benzoate NF | Buffering Agent | 0.049 | 0.050 |
| Glycerin USP* | Vehicle for ethyl | 0.589 | 0.600 |
| | lauroyl arginate | | |
| | raw material | | |
| Total | | 100.00 | |

*Ethyl lauroyl arginate HCl (LAE), which is provided in the raw material as a 20% w/w solution in glycerin under the trade name of Aminat G.

The mouthrinse from Table 1 was prepared in a suitable covered container according to the following procedure:

a. Added Alcohol and water (60:40), Aminat G, Poloxamer 407, flavor and benzoic acid into the container. Mixed until homogeneous.

b. In above container, added Sorbitol and USP Water. Mix until homogeneous.

c. In above container, added Sodium Benzoate and Sucralose. Mix until homogeneous.

d. Checked that pH is 4.2+/−0.1 at room temperature and adjusted with minimal NaOH or HCl (if necessary).

e. Recorded pH

f. Filled into PET bottle

LAE bioavailability was assessed by the Disc Retention Assay (DRA) outlined below.

### Procedure for Determining Bioavailability

Based on L. A. Bacca, M. J. Doyle, D. M. Macksood, A. C. Lanzalaco. Availability Testing of Cetylpyridinium Chloride Mouthrinses. Research presented at the 75th General Session of the IADR; Orlando, Fla. Mar. 19-23, 1997.

### Materials

Cellulose Filter disks (Schleicher and Schuell #740-E)

24-well culture plate (Corning 3526)

Tweezers

Vacuum pulled filtration system

Water

0.1% Acid Fuschin dye solution (w/v)

20% p-toluenesulfonic acid solution (w/v)

25 mm Syringe Filter w/0.45 μm Nylon Membrane

10 mL Control Syringe

1 N HCl

Spectrophotometer (Molecular Devices—SpectraMAX 190)

### Method

1. Placed a cellulose filter disk into a clean well using tweezers to prevent contamination of the disk by contact with skin tissue.

2. Pipetted a 60 μl aliquot of the test sample onto the disk and allowed to stand for 1 minute.

3. Transferred the filter disk to a standard vacuum pulled filtration system.

4. Suction filtered a 15 ml water wash through the treated cellulose disk to remove the non-bound components of the treatment solution.

5. Suction filtered a 5 ml aliquot of a 0.1% acid fuschin dye solution (w/v) through the treated cellulose disk, followed by three 15 ml water rinses to remove the excess dye.

6. Removed the cellulose disk from the filtration system and place it into a clean test tube containing 4 ml of a 20% p-toluenesulfonic acid solution (w/v).

7. Vortexed the disk in the test tube for 30 seconds.

8. Poured solution into a clean well, aspirate solution with 10 mL control syringe.

9. Syringe filtered the resulting solution through a 0.45 μm filter into a clean test tube.

10. Added 90 μl of 1 N HCl to the filtered solution. Vortexed solution.

11. Dispensed 200 μl of each solution into a column of a 3595 96-well plate.

12. Read absorbance spectrophotometrically at 545 nm.

The samples were run three times, and the results represented the average of the three runs.

$$\% BA = \frac{Calculated Available LAE}{Formulated level LAE} \times 100$$

Bioavailability (BA) was measured at each step of the batching process and the results are summarized in Table 2. Briefly, the full formula bioavailability is 43.6% as measured.

TABLE 2

| Description | Mean BA ± S.E. |
|---|---|
| Water, Aminat G and Ethanol | 78.1 ± 3.4 |
| Water, Aminat G, Ethanol and | 80.5 ± 5.9 |
| Poloxamer 407 | |
| Water, Aminat G, Ethanol, Poloxamer | 57.5 ± 6.3 |
| 407 and Flavor | |
| Water, Aminat G, Ethanol, Poloxamer | 54.0 ± 5.4 |
| 407, Flavor and Benzoic Acid | |
| Water, Aminat G, Ethanol, Poloxamer | 46.3 ± 10.8 |
| 407, Flavor, Benzoic Acid and Sorbitol | |
| Water, Aminat G, Ethanol, Poloxamer | 47.3 ± 4.3 |
| 407, Flavor, Benzoic Acid, Sorbitol and | |
| Sodium Benzoate | |
| Water, Aminat G, Ethanol, Poloxamer | 43.6 ± 9.3 |
| 407, Flavor, Benzoic Acid, Sorbitol, | |
| Sodium Benzoate and Sucralose | |
| Water, Ethanol, Poloxamer 407, Flavor, | 14.9 ± 9.8 |
| Benzoic Acid, Sorbitol, Sodium | |
| Benzoate and Sucralose (no LAE) | |

### Example 2

The effects of different surfactants on LAE bioavailability were evaluated and findings are summarized in Table 3. All Formulations were made using the same procedure as defined in Example 1 with the only difference being the surfactant of choice and in what concentration the surfactant was added.

TABLE 3

| | Concentration in | | |
|---|---|---|---|
| | Formulation | BA ± SE | BA ± SE |
| Surfactant of Choice | (w/v) | with LAE | without LAE |
| Poloxamer 407 | 0.5000% | 35.9 ± 5.2 | Not available |
| Poloxamer 188 | 0.5000% | 46.3 ± 7.5 | Not available |
| Polysorbate 20 | 0.5000% | 33.7 ± 2.8 | Not available |
| Cremophor | 0.5000% | 30.7 ± 3.9 | Not available |
| Cocamidopropyl Betaine | 0.5000% | 86.1 ± 2.8 | 27.6 ± 15.0 |

Surprisingly, it was found that Cocamidopropyl Betaine (CAPB) did not negatively impact bioavailability.

### Example 3

Table 4 below shows the bioavailability results of CAPB (a distribution of molecular weights ranging from C8 to C18) at different pHs found in mouthrinses, a Betaine surfactant with specified alkyl chain lengths (C12 and C16) and a formulation containing CAPB found in Colgate patent U.S. Pat. No. 8,287,843B2. All formulations (with exception of formulation disclosed in U.S. Pat. No. 8,287,843B2) were made using the same procedure as defined in Example 1 with the only difference being the surfactant of choice and in what concentration the surfactant was added.

TABLE 4

| | Concentration | | |
|---|---|---|---|
| | in | | BA ± SE |
| | Formulation | BA ± SE with | without |
| Surfactant of Choice | (w/v) | LAE | LAE |
| Cocamidopropyl | 0.3000% | 139.7 ± 9.1** | Not available |
| Betaine (pH 4.2) | | | |
| Cocamidopropyl | 0.3000% | 57.5 ± 19.5 | 22.7 ± 9.4 |
| Betaine (pH 2.0) | | | |
| Cocamidopropyl | 0.3000% | 126.4 ± 0.8** | 19.3 ± 5.5 |
| Betaine (pH 3.5) | | | |
| Cocamidopropyl | 0.3000% | 118.3 ± 5.4** | 18.9 ± 12.0 |
| Betaine (pH 5.5) | | | |
| Cocamidopropyl | 0.3000% | 70.9 ± 8.5 | 22.7 ± 20.6 |
| Betaine (pH 7.0) | | | |
| Lauramidopropyl | 0.8571% | 98.3 ± 9.0 | 17.2 ± 7.8 |
| Betaine, 35% active | | | |
| (pH 4.2) | | | |
| Cetyl Betaine, 30.88% | 0.9715% | 169.3 ± 6.5** | 42.4 ± 7.5 |
| active (pH 4.2) | | | |
| Cocamidopropyl | 0.8000%/ | 21.5 ± 10.6 | 20.8 ± 4.5 |
| Betaine/Polysorbate-20* | 0.8000% | | |

*formulation disclosed in Colgate patent U.S. Pat. No. 8,287,843B2.
**Calculated available LAE level is higher than formulated due to limits of detection of this assay

### Example 4

Samples described in this invention were also analyzed using a Zetasizer Nano ZS DLS instrument (Malvern Instruments, Inc., Southborough, Mass.) operating at 25.0° C. Samples must yield a minimum count rate of 100,000 counts per second (cps) for accurate determination of micelle hydrodynamic radius (dH) and micelle size distribution. For samples with count rates below this minimum, the sample concentration may be gradually increased (i.e. diluted less) until the minimum count rate is achieved, or in some cases, the sample may be run in neat form. Values of micelle dH and the micelle size distribution are calculated using the Dispersion Technology Software (DTS) v4.10 package (Malvern Instruments Inc., Southborough, Mass.), which calculates the Z-average micelle dH according to the ISO13321 test method. Values of average micelle dH are reported herein as the Z-average micelle dH. The reported values of micelle dH are the average of at least two individual measurement runs. The intensity distribution of micelle size calculated by the DTS software is used to calculate the fraction of micelles having values of dH under a given size limit. Particle size measurements for Poloxamer, CAPB at different pHs with and without LAE are show in Table 5. The change in average micelle size suggests a potential interaction between the two surfactants.

TABLE 5

| | Average Micelle Size | |
|---|---|---|
| Sample | Without LAE | With LAE |
| Poloxamer 407, pH 4.2 | 88.02 nm | 62.41 nm |
| CAPB, pH 4.2 | 19.23 nm | 14.79 nm |
| Colgate patent U.S.Pat. No. 8,287,843B2 | 14.63 nm | 15.56 nm |
| formulation, pH 5.0 | | |

### Example 5

A concentrated composition is prepared having composition shown in Table 6 and made as described in Example 1 except that no additional purified water is added at the start.

TABLE 6

| | | Quantity in |
|---|---|---|
| | | finished product |
| Component | Function | (% w/w) |
| Purified Water USP | Vehicle | 0.000 |
| Sorbitol Solution 70% USP | Humectant | 49.625 |
| Ethanol USP (190 proof) | Solvent | 45.695 |
| Flavor - Mint | Flavor | 0.364 |
| Poloxamer 407 NF | Surfactant | 0.619 |
| Ethyl Lauroyl Arginate | Anti-plaque | 0.372 |
| HCl (LAE)* | adhesion agent | |
| Benzoic Acid USP | Buffering Agent | 0.298 |
| Sucralose NF | Sweetener | 0.149 |
| Sodium Benzoate NF | Buffering Agent | 0.124 |
| Glycerin USP* | Vehicle for ethyl | 1.489 |
| | lauroyl arginate raw | |
| | material | |
| Cocamidopropyl Betaine | Surfactant | 1.264 |
| Total | | 100.00 |

What is claimed is:

1. A dispensing device comprising a container containing an oral care composition and a dispensing assembly associated with the container for dispensing the composition from the device through an orifice, said composition comprising:
a) one or more N^{α}-acyl acidic amino acid ester salts represented by the formula I: 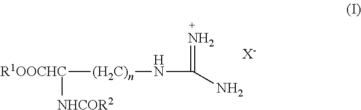
wherein R¹ is an alkyl group having from 1 to 8 carbons, R² is an alkyl group having from 6 to 30 carbons, n is from 1 to 6 and X⁻ is an anion;
b) a zwitterionic surfactant;
c) at least 40% v/v of the total composition of an alcohol represented by R³—OH, wherein R³ is an alkyl group having from 2 to 6 carbons; and
d) an oily component comprising at least one essential oil, at least one flavor oil, or a combination of two or more thereof;
wherein the composition comprises less than 30% v/v of water.

2. The device of claim 1 wherein said one or more N^{α}-acyl acidic amino acid ester salts represented by the formula I is selected from the group consisting of: chloride salts of N^{α}-cocoyl-L-arginine ethyl ester, N^{α}-lauryl-L-arginine ethyl ester, and N^{α}-lauryl-L-arginine iso-propyl ester; DL-pyrrolidone carboxylate salts of N^{α}-cocoyl-L-arginine ethyl ester, N^{α}-lauryl-L-arginine ethyl ester, and N^{α}-lauryl-L-arginine iso-propyl ester; and combinations of two or more thereof, and wherein said zwitterionic surfactant is a betaine surfactant selected from the group consisting of alkyldimethyl betaines, amidobetaines, and combinations of two or more thereof.

3. The device of claim 2 wherein said composition comprises at least 50% v/v of the total composition of an alcohol represented by R³—OH, wherein R³ is an alkyl group having from 2 to 6 carbons; and wherein said oily component comprises at least one essential oil selected from the group consisting of menthol, thymol, eucalyptol, methyl salicylate, and combinations of two or more thereof.

4. The device of claim 3, wherein said composition comprises at least 60% v/v of the total composition of an alcohol represented by R³—OH, wherein R³ is an alkyl group having from 2 to 6 carbons.

5. The device of claim 4 wherein R³ is the same as R′.

6. The device of claim 3 wherein said dispensing assembly comprises a pump spraying assembly.

7. The device of claim 6 wherein said dispensing assembly comprises a manual pump spraying assembly.

8. The device of claim 3 wherein said dispensing assembly comprises one or more aerosols for dispensing said composition.

9. A method of inhibiting plaque in an oral cavity comprising using said device of claim 1 to spray said oral care composition into the oral cavity.

10. A method of inhibiting plaque in an oral cavity comprising using said device of claim 6 to spray said oral care composition into the oral cavity.

11. A method of treating the oral cavity comprising spraying into the oral cavity a composition comprising
a) one or more N^{α}-acyl acidic amino acid ester salts represented by the formula I: 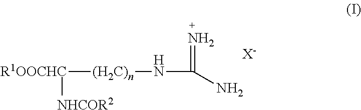
wherein R¹ is an alkyl group having from 1 to 8 carbons, R² is an alkyl group having from 6 to 30 carbons, n is from 1 to 6 and X⁻ is an anion;
b) a zwitterionic surfactant;
c) at least 40% v/v of the total composition of an alcohol represented by R³—OH, wherein R³ is an alkyl group having from 2 to 6 carbons; and
d) an oily component comprising at least one essential oil, at least one flavor oil, or a combination of two or more thereof;
wherein the composition comprises less than 30% v/v of water.
